(19) United States
(12) Patent Application Publication
(10) Pub. No.: US 2026/0360550 A1
(43) Pub. Date: Nov. 12, 2026

(54) INFORMATION PROCESSING DEVICE, SYSTEM, PROGRAM, AND METHOD FOR INFERRING DIAGNOSIS-RELATED INFORMATION RELATING TO PSYCHIATRIC/NEUROLOGICAL DISEASE OF SUBJECT

(71) Applicant: FrontAct Co., Ltd., Chuo-ku, Tokyo (JP)

(72) Inventors: Seiji HORI, Chuo-ku, Tokyo (JP); Naomi KAMIOKA, Chuo-ku, Tokyo (JP); Takashi KOMANO, Chuo-ku, Tokyo (JP); Yasushi OCHIAI, Chuo-ku, Tokyo (JP); Kazuki KASAI, Setagaya-ku, Tokyo (JP)

(21) Appl. No.: 19/130,053

(22) PCT Filed: Nov. 14, 2023
(86) PCT No.: PCT/JP2023/040919

§ 371 Date: May 14, 2025

(30) Foreign Application Priority Data
Nov. 14, 2022 (JP) 2022-181738

(51) Int. Cl. G16H 50/30; G06T 13/40; G16H 40/67; G16H 80/00

(52) U.S. Cl. CPC G16H 50/30; G06T 13/40; G16H 40/67; G16H 80/00

(57) ABSTRACT

A diagnosis unit estimates diagnosis-related information from diagnosis parameter in at least two of a first to a third cases. A output unit outputs the diagnosis-related information estimated by the diagnosis unit to the outside of an information processing device. The diagnosis parameter includes word-and-action information of the subject. The word-and-action information of the subject includes at least one of speech information and a behavior history of the subject. The first case is a case in which the subject is outside a specific facility where the subject and a doctor meet face-to-face for a medical examination of the neuropsychiatric disorder by the doctor, and in which the subject is not accessing a virtual space. The second case is a case in which the subject and the doctor are meeting face-to-face in the specific facility. The third case is a case in which the subject is accessing the virtual space.

### TECHNICAL FIELD

The present invention relates to a system, a program and a method for estimating diagnosis-related information about a neuropsychiatric disorder of a subject.

### BACKGROUND ART

A configuration that estimates diagnosis-related information about a neuropsychiatric disorder of a subject has been conventionally known. For example, Japanese Patent Laying-Open No. 2020-149234 (PTL 1) discloses a device that estimates whether a user has dementia, based on behavior data of the user having the behavior of the user chronologically recorded thereon, using a learned model that has learned a relationship between the behavior data and a degree of dementia.

### CITATION LIST

### Patent Literature

PTL 1: Japanese Patent Laying-Open No. 2020-149234

### SUMMARY OF INVENTION

### Technical Problem

A subject having a neuropsychiatric disorder does not always perform the behavior characteristic of the neuropsychiatric disorder (characteristic behavior). For example, the subject may suppress the characteristic behavior in front of people so as not to allow others to know that the subject has the neuropsychiatric disorder. On the other hand, the subject can perform the characteristic behavior for a specific purpose (e.g., a purpose of attracting attention of those around the subject, a purpose of obtaining monetary gain, or a purpose of evading an obligation), although the subject does not have the neuropsychiatric disorder. Therefore, behavior data having the behavior of the subject simply chronologically recorded thereon may not include the characteristic behavior of the subject having the neuropsychiatric disorder, whereas the behavior data may include the characteristic behavior created or exaggerated by the subject that does not have the neuropsychiatric disorder. Such behavior data may cause deviation between evaluation of the neuropsychiatric disorder of the subject and an objective state of the subject. However, PTL 1 does not take the deviation into consideration.

The present invention has been made to solve the above-described problem, and an object thereof is to assist objective diagnosis and state evaluation relating to a neuropsychiatric disorder of a subject.

### Solution to Problem

An information processing device according to an aspect of the present invention estimates diagnosis-related information about a neuropsychiatric disorder of a subject. The information processing device includes: a diagnosis unit; and an output unit. The diagnosis unit estimates the diagnosis-related information from a diagnosis parameter in at least two of a first case, a second case and a third case. The output unit outputs the diagnosis-related information estimated by the diagnosis unit to the outside of the information processing device. The diagnosis parameter includes word-and-action information of the subject. The word-and-action information of the subject includes at least one of speech information and a behavior history of the subject. The first case is a case in which the subject is outside a specific facility where the subject and a doctor meet face-to-face for a medical examination of the neuropsychiatric disorder by the doctor, and in which the subject is not accessing a virtual space. The second case is a case in which the subject and the doctor are meeting face-to-face in the specific facility. The third case is a case in which the subject is accessing the virtual space.

A system according to another aspect of the present invention estimates diagnosis-related information about a neuropsychiatric disorder of a subject. The system includes: at least one word-and-action monitoring device; a terminal device; and an information processing device. The at least one word-and-action monitoring device acquires word-and-action information of the subject. The information processing device estimates the diagnosis-related information from a diagnosis parameter and transmits the diagnosis-related information to the terminal device. The diagnosis parameter includes the word-and-action information of the subject acquired by the at least one word-and-action monitoring device. The word-and-action information of the subject includes at least one of speech information and a behavior history of the subject. The diagnosis parameter includes word-and-action information of the subject in at least two of a first case, a second case and a third case. The first case is a case in which the subject is outside a specific facility where the subject and a doctor meet face-to-face for a medical examination of the neuropsychiatric disorder by the doctor, and in which the subject is not accessing a virtual space. The second case is a case in which the subject and the doctor are meeting face-to-face in the specific facility. The third case is a case in which the subject is accessing the virtual space.

A program according to another aspect of the present invention is a program for estimating diagnosis-related information about a neuropsychiatric disorder of a subject. By being executed by a processor, the program estimates the diagnosis-related information from a diagnosis parameter in at least two of a first case, a second case and a third case, and transmits the diagnosis-related information to a terminal device. The diagnosis parameter includes word-and-action information of the subject. The word-and-action information of the subject includes at least one of speech information and a behavior history of the subject. The first case is a case in which the subject is outside a specific facility where the subject and a doctor meet face-to-face for a medical examination of the neuropsychiatric disorder by the doctor, and in which the subject is not accessing a virtual space. The second case is a case in which the subject and the doctor are meeting face-to-face in the specific facility. The third case is a case in which the subject is accessing the virtual space.

A method according to another aspect of the present invention estimates diagnosis-related information about a neuropsychiatric disorder of a subject. The method includes: estimating the diagnosis-related information from a diagnosis parameter in at least two of a first case, a second case and a third case; and outputting the diagnosis-related information to a terminal device. The diagnosis parameter includes word-and-action information of the subject. The word-and-action information of the subject includes at least one of speech information and a behavior history of the subject. The first case is a case in which the subject is outside a specific facility where the subject and a doctor meet face-to-face for a medical examination of the neuropsychiatric disorder by the doctor, and in which the subject is not accessing a virtual space. The second case is a case in which the subject and the doctor are meeting face-to-face in the specific facility. The third case is a case in which the subject is accessing the virtual space.

### Advantageous Effects of Invention

According to the system, the program and the method of the present invention, the diagnosis parameter includes the word-and-action information of the subject in at least two of the first case, the second case and the third case, and thus, objective diagnosis and state evaluation relating to the neuropsychiatric disorder of the subject can be assisted.

### BRIEF DESCRIPTION OF DRAWINGS

FIG. 1 is a block diagram showing a configuration of a neuropsychiatric disorder diagnosis system according to a first embodiment.

FIG. 2 is a block diagram showing a functional configuration of an information processing device in FIG. 1.

FIG. 3 is a flowchart showing an example of a flow of a process performed by the information processing device in FIG. 2.

FIG. 4 is a diagram showing an example of a hardware configuration of each of the information processing device and a terminal device in FIG. 2.

FIG. 5 is a block diagram showing a configuration of a neuropsychiatric disorder diagnosis system according to a second embodiment.

FIG. 6 is a block diagram showing a functional configuration of an information processing device in FIG. 5.

FIG. 7 is a flowchart showing an example of a flow of a process performed by the information processing device in FIG. 6.

### DESCRIPTION OF EMBODIMENTS

Hereinafter, embodiments of the present invention will be described in detail with reference to the drawings, in which the same or corresponding portions are denoted by the same reference characters and description thereof will not be repeated in principle.

### First Embodiment

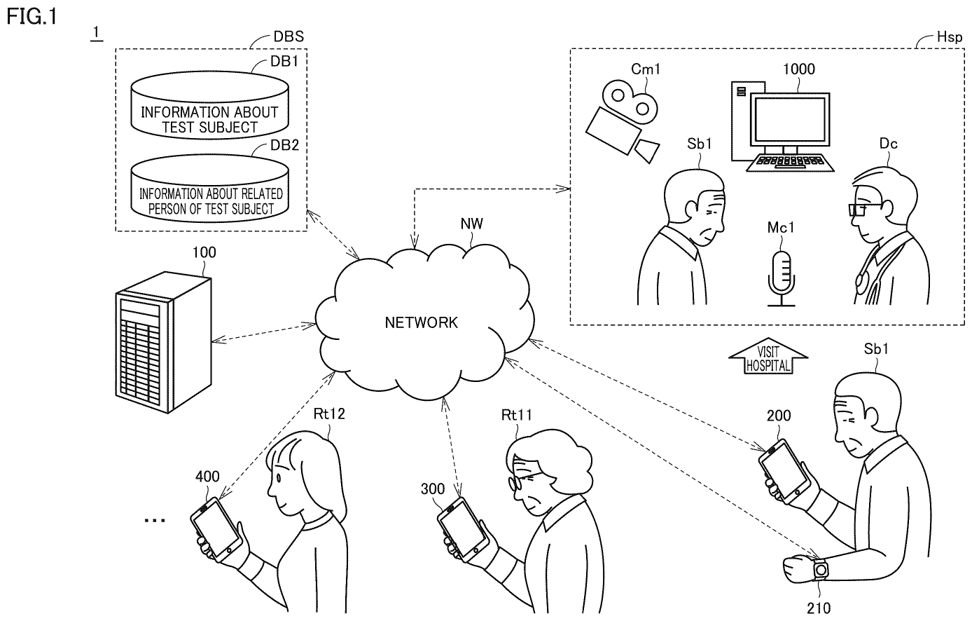

FIG. 1 is a block diagram showing a configuration of a neuropsychiatric disorder diagnosis system 1 according to a first embodiment. Neuropsychiatric disorder diagnosis system 1 estimates diagnosis-related information about a neuropsychiatric disorder of a test subject Sb1 (a patient or a subject). The diagnosis-related information includes the presence or absence and a state, prodromal symptoms, a pre-symptomatic state, and a relapse sign of the neuropsychiatric disorder. The neuropsychiatric disorder in the present disclosure includes schizophrenia, bipolar disorder (manic depression), depression, anxiety disorder, panic disorder, obsessive-compulsive disorder, post-traumatic stress disorder (PTSD), dementia, autism spectrum disorder, attention-deficit hyperactivity disorder, neurodevelopmental disorder, eating disorder, epilepsy, addiction or the like. The present disclosure is applicable to any neuropsychiatric disorder. The present disclosure is preferably applied to diagnosis of the neuropsychiatric disorder including schizophrenia, bipolar disorder, depression and/or dementia, and is particularly suitably applied to diagnosis of the neuropsychiatric disorder including schizophrenia.

As shown in FIG. 1, neuropsychiatric disorder diagnosis system 1 includes an information processing device 100, a terminal device 200 (word-and-action monitoring device) of test subject Sb1, a terminal device 210, a terminal device 300 of a spouse Rt11 of test subject Sb1, a terminal device 400 of a visiting nurse Rt12 of test subject Sb1, a terminal device 1000 in a hospital Hsp, a camera Cm1 (word-and-action monitoring device), a microphone Mc1 (word-and-action monitoring device), and a database server DBS. Terminal devices 200, 210, 300, 400, and 1000, camera Cm1, microphone Mc1, and database server DBS are connected to information processing device 100 through a network NW. Network NW includes, for example, the Internet, a local area network (LAN) or a cloud system.

Test subject Sb1 visits hospital Hsp (specific facility) for a medical examination of the neuropsychiatric disorder by a doctor Dc. Word-and-action information of test subject Sb1 (at least one of speech information and behavior history information of test subject Sb1) when test subject Sb1 and doctor Dc are meeting face-to-face in hospital Hsp is recorded using camera Cm1 and microphone Mc1, and is transmitted from camera Cm1 and microphone Mc1 to information processing device 100 together with time information (time stamp). A cycle of sampling and a timing of acquisition of the word-and-action information of test subject Sb1 using camera Cm1 and microphone Mc1 may be arbitrarily set.

From image data acquired by camera Cm1, the presence or absence of an abnormal behavior and a type of the abnormal behavior (e.g., leg shaking or head scratching) are, for example, acquired as information about the behavior history of test subject Sb1. In addition, from this image data, a type of facial expression (e.g., joy or anger) and a manner of change in facial expression (e.g., steady clenching of a back teeth) of test subject Sb1 are, for example, acquired as information about a neuropsychiatric state (e.g., a psychiatric state or a state of a psychiatric disorder) of test subject Sb1. From speech data acquired using microphone Mc1, text information of conversation with doctor Dc is acquired as the speech information. In addition, from this speech data, an amount of utterance, a speech information content, a state of conversation, a tone (intonation) of the speech information, or a speech frequency is, for example, acquired as the information about the neuropsychiatric state of test subject Sb1.

Doctor De measures biological information (e.g., a body temperature (°C), a heart rate (bpm) or a heart rate variability (HRV) (ms)) of test subject Sb1 that is having the medical examination, and inputs the biological information to terminal device 1000. Terminal device 1000 transmits the biological information of test subject Sb1 to information processing device 100 together with the time information. Terminal device 1000 includes, for example, a personal computer, a tablet or a smartphone.

Even when test subject Sb1 has the neuropsychiatric disorder, test subject Sb1 does not always perform the abnormal behavior. For example, test subject Sb1 may suppress the abnormal behavior in front of doctor Dc in order to conceal the fact that test subject Sb1 has the neuropsychiatric disorder from doctor Dc. On the other hand, test subject Sb1 can perform the abnormal behavior for a specific purpose in front of doctor Dc, although test subject Sb1 does not have the neuropsychiatric disorder. Therefore, there is a possibility that the behavior history of test subject Sb1 in front of doctor De does not include the abnormal behavior of test subject Sb1 having the neuropsychiatric disorder, whereas the behavior history of test subject Sb1 in front of doctor Dc may include the abnormal behavior created or exaggerated by test subject Sb1 that does not have the neuropsychiatric disorder. Such behavior history may cause deviation between diagnosis of the neuropsychiatric disorder of the subject by doctor Dc and an objective state of test subject Sb1. In addition, among the neuropsychiatric disorders, dementia can be diagnosed to some extent by not only a medical interview but also a neuropsychological test, a brain imaging test and the like. However, particularly regarding schizophrenia, there is no definitive test method, and thus, there is a need for a method for assisting diagnosis based on objective data and the like of test subject Sb1.

Thus, neuropsychiatric disorder diagnosis system 1 estimates the diagnosis-related information about the neuropsychiatric disorder of test subject Sb1 from a diagnosis parameter including word-and-action information in front of doctor Dc and word-and-action information of test subject Sb1 in the real world outside hospital Hsp. An objective state of test subject Sb1 is more likely to be expressed unconsciously in the word-and-action information of test subject Sb1 in the real world outside hospital Hsp than that in front of doctor Dc. According to neuropsychiatric disorder diagnosis system 1, objective diagnosis and state evaluation relating to the neuropsychiatric disorder of test subject Sb1 can be assisted. The diagnosis parameter may include diagnosis-related information acquired in the past. Hereinafter, the diagnosis parameter of test subject Sb1 when test subject Sb1 and doctor Dc are meeting face-to-face in hospital Hsp will also be referred to as in-hospital information of test subject Sb1. The in-hospital information of test subject Sb1 is not limited to information at the present time when test subject Sb1 and doctor Dc are meeting face-to-face, and may include a diagnosis parameter and diagnosis-related information when test subject Sb1 and doctor Dc met face-to-face in the past. The in-hospital information of test subject Sb1 may also include a diagnosis parameter and diagnosis-related information in the past acquired in a hospital different from the hospital to which doctor Dc currently belongs.

Terminal device 200 of test subject Sb1 acquires word-and-action information of test subject Sb1 in daily life, and transmits the word-and-action information to information processing device 100 together with time information. “Daily life” refers to a case in which test subject Sb1 is outside hospital Hsp. Terminal device 200 includes, for example, a smartphone, a tablet and a personal computer. The information about the behavior history acquired by terminal device 200 includes, for example, an operation time of terminal device 200 per day, a purchase history (e.g., the number of purchases and a total purchase amount) in Internet shopping per week, a movement range of terminal device 200, or a text input to a search engine, a social networking service (SNS) or an electronic mail. The speech information acquired by terminal device 200 includes, for example, text information of speech conversation of test subject Sb1. Hereinafter, the diagnosis parameter of test subject Sb1 in daily life will also be referred to as out-of-hospital information. A cycle of sampling and a timing of acquisition of the word-and-action information using terminal device 200 of test subject Sb1 may be arbitrarily set.

Terminal device 210 of test subject Sb1 includes a biological information sensor (e.g., a pulse sensor). Terminal device 210 acquires the biological information of test subject Sb1 through the biological information sensor, and transmits the biological information to information processing device 100 together with time information. Terminal device 210 includes, for example, a wearable smart watch. A cycle of sampling and a timing of acquisition of the biological information using terminal device 210 of test subject Sb1 may be arbitrarily set.

Database server DBS includes databases DB1 and DB2. Information about test subject Sb1 is registered in database DB1. The information about test subject Sb1 includes, for example, personal information, a medical history, and identification information (e.g., an ID, a password, or physical characteristics for biometric authentication such as a fingerprint, a voiceprint, a vein pattern, a retina, and an iris) for authentication of test subject Sb1. Information about a related person of test subject Sb1 is registered in database DB2. The information about the related person of test subject Sb1 includes, for example, personal information and authentication information of the related person. The related person of test subject Sb1 includes, for example, a family such as spouse Rt11 of test subject Sb1, a colleague, visiting nurse Rt12, or a social worker.

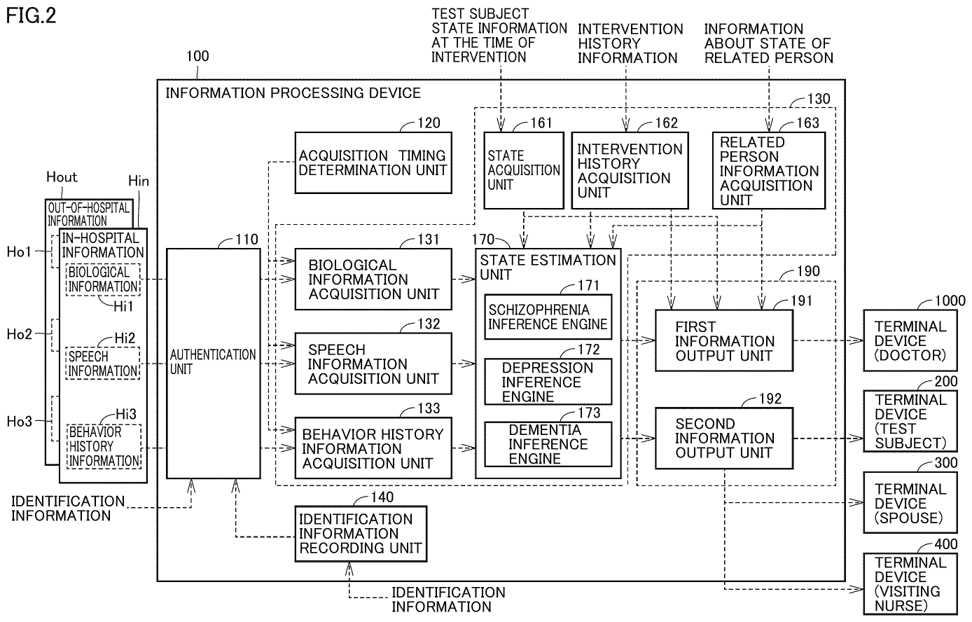

FIG. 2 is a block diagram showing a functional configuration of information processing device 100 in FIG. 1. As shown in FIG. 2, information processing device 100 receives in-hospital information Hin and out-of-hospital information Hout. In-hospital information Hin includes biological information Hi1, speech information Hi2 and behavior history information Hi3 of test subject Sb1. Out-of-hospital information Hout includes biological information Ho1, speech information Ho2 and behavior history information Ho3 of test subject Sb1.

Information processing device 100 includes an authentication unit 110, an acquisition timing determination unit 120, a diagnosis unit 130, an identification information recording unit 140, and an output unit 190. Diagnosis unit 130 includes a biological information acquisition unit 131, a speech information acquisition unit 132, a behavior history information acquisition unit 133, a state acquisition unit 161, an intervention history acquisition unit 162, a related person information acquisition unit 163, and a state estimation unit 170. Output unit 190 includes a first information output unit 191 and a second information output unit 192.

The identification information of test subject Sb1 acquired from database server DBS is registered in advance in identification information recording unit 140. Authentication unit 110 determines whether the identification information registered in identification information recording unit 140 (registered identification information) matches identification information input from terminal device 200 of test subject Sb1 (input identification information), and outputs a result of determination to biological information acquisition unit 131, speech information acquisition unit 132 and behavior history acquisition unit 150.

Acquisition timing determination unit 120 determines a timing of acquisition of the diagnosis parameter and outputs the timing of acquisition to state estimation unit 170. The timing of acquisition of the diagnosis parameter may be, for example, a timing of occurrence of a predetermined trigger event. The trigger event includes, for example, arrival of the predetermined time or capturing of a trigger word. The timing of acquisition of the diagnosis parameter may be random.

When the input identification information matches the registered identification information and when the timing of acquisition of the diagnosis parameter arrives, biological information acquisition unit 131 acquires biological information at the timing of acquisition from the received data, and outputs the biological information to state estimation unit 170. Biological information acquisition unit 131 may acquire, for example, an amount of utterance, a tone, speech, object description information, a response to a question from doctor Dc, a posture, a facial expression, and line-of-sight information of test subject Sb1 from the received data, and output these to state estimation unit 170.

When the input identification information matches the registered identification information and when the timing of acquisition of the diagnosis parameter arrives, speech information acquisition unit 132 acquires, for example, conversation information of test subject Sb1 at the timing of acquisition, text information obtained by converting the conversation information into text data, and an anger word included in the text information from the received data as the speech information of test subject Sb1, and outputs these to state estimation unit 170.

When the input identification information matches the registered identification information and when the timing of acquisition of the diagnosis parameter arrives, behavior history information acquisition unit 133 acquires, for example, an operation time of terminal device 200 of test subject Sb1 at the timing of acquisition, a purchase history, a search word, a content input to the SNS, and a movement range of terminal device 200 as the behavior history information of test subject Sb1, and outputs these to state estimation unit 170.

Personal authentication by authentication unit 110 makes it possible to prevent data about a person different from test subject Sb1 from being used for diagnosis of the neuropsychiatric disorder of test subject Sb1. In addition, setting the timing of acquisition of the diagnosis parameter makes it possible to use the diagnosis parameter under a situation in which the objective state of test subject Sb1 is likely to be expressed and to prevent the conscious abnormal behavior of test subject Sb1. As a result, the objectivity of diagnosis of the neuropsychiatric disorder of test subject Sb1 by information processing device 100 can be further increased.

When intervention is performed by the related person (e.g., Rt11 or Rt12) of test subject Sb1 for the purpose of care or independence support of test subject Sb1, state acquisition unit 161 acquires intervention information input by the intervening related person (intervenor) through his/her own terminal device (e.g., 300 or 400) together with time information, and outputs the intervention information to state estimation unit 170 and first information output unit 191. The intervention information includes, for example, a neuropsychiatric state of test subject Sb1 when the related person intervenes in the behavior of test subject Sb1, an increase or decrease in frequency of going out, information about the relationship with friends, a content of conversation, a medicine-taking situation (e.g., a remaining amount or a management state of medicine), information about whether a home-visit care service (home-visit care or home-visit nursing) has been provided to test subject Sb1, information about a neuropsychiatric state of test subject Sb1 when the home-visit care service has been provided, and information about a trigger event when the neuropsychiatric state of test subject Sb1 improves or worsens.

Intervention history acquisition unit 162 acquires history information (intervention history information) about intervention in test subject Sb1 by the intervenor from the terminal device of the intervenor together with time information, and outputs the history information to state estimation unit 170 and the first information output unit. The history information includes, for example, a type of the intervenor (e.g., a spouse, a social worker, a caregiver, or a visiting nurse).

Related person information acquisition unit 163 acquires information about a state of the related person (e.g., Rt11 or Rt12) of test subject Sb1 from the terminal device of the related person or database server DBS, and outputs the information to state estimation unit 170 and first information output unit 191. The information about the state of the related person of test subject Sb1 includes information about a neuropsychiatric disorder of the related person.

State estimation unit 170 estimates the diagnosis-related information about the neuropsychiatric disorder of test subject Sb1 from the diagnosis parameter acquired from at least one of speech information acquisition unit 132 and behavior history information acquisition unit 133, and outputs the diagnosis-related information to first information output unit 191 and second information output unit 192. The diagnosis parameter includes the word-and-action information of test subject Sb1 when test subject Sb1 and doctor Dc are meeting face-to-face in hospital Hsp, and the word-and-action information of test subject Sb1 in the real world outside hospital Hsp. The diagnosis parameter may further include the biological information of test subject Sb1 acquired by biological information acquisition unit 131, the intervention information acquired by state acquisition unit 161, the intervention history information acquired by intervention history acquisition unit 162, and the information about the state of the related person of test subject Sb1 acquired by related person information acquisition unit 163.

Using, for example, the information such as the amount of utterance, the tone, the speech, the response to the question, the posture, the facial expression, and the line-of-sight information of test subject Sb1, which are included in the intervention information, state estimation unit 170 determines the diagnosis-related information about the neuropsychiatric disorder of test subject Sb1 based on a difference from a general healthy person or a difference from history data of the test subject in the past.

State estimation unit 170, for example, compares the operation time of the terminal device of test subject Sb1, the purchase history, the search word, the content of the SNS, and the behavior range history information, which are included in the behavior history information of test subject Sb1, with characteristic statistical data or behavior relating to the neuropsychiatric disorder of test subject Sb1, and determines whether the words and actions of test subject Sb1 include the abnormal behavior.

State estimation unit 170, for example, compares the conversation information of test subject Sb1 and the text information obtained by converting the conversation information into text data, which are included in the speech information, with characteristic statistical data or behavior relating to the neuropsychiatric disorder of test subject Sb1, and determines whether the words and actions of test subject Sb1 include the abnormal behavior.

State estimation unit 170 includes at least one of a schizophrenia inference engine 171, a depression inference engine 172 and a dementia inference engine 173. Schizophrenia inference engine 171 infers diagnosis-related information about schizophrenia from the diagnosis parameter Schizophrenia inference engine 171 can use a model for determining doubtfulness, severity, a sign of relapse or the like of schizophrenia from the word-and-action information of test subject Sb1, for example. Depression inference engine 172 infers diagnosis-related information about depression from the diagnosis parameter. Depression inference engine 172 can use a model for determining doubtfulness, severity and the like of depression by using the biological information and the behavior history acquired in and/or outside the hospital, for example. Dementia inference engine 173 infers diagnosis-related information about dementia from the diagnosis parameter. State estimation unit 170 may infer the pieces of diagnosis-related information about the disorders using schizophrenia inference engine 171, depression inference engine 172 and dementia inference engine 173, respectively, or may integrate any of the pieces of diagnosis-related information and infer the diagnosis-related information about the neuropsychiatric disorder of test subject Sb1.

State estimation unit 170, for example, compares a reference feature amount, which is prepared in advance to determine the neuropsychiatric disorder and indicates that test subject Sb1 has the neuropsychiatric disorder, with the diagnosis parameter including out-of-hospital information Hout, in-hospital information Hin, the intervention history information, and the related person information of test subject Sb1, and quantitatively determines a tendency of the neuropsychiatric disorder of test subject Sb1 based on a distance between the reference feature amount and the diagnosis parameter in a feature amount space. State estimation unit 170 may determine the state of test subject Sb1 and capture a change in which test subject Sb1 is interested, by analyzing a difference and a degree of deviation between word-and-action information of test subject Sb1 when test subject Sb1 is outside hospital Hsp and when test subject Sb1 is not accessing the virtual space and word-and-action information of test subject Sb1 when test subject Sb1 and doctor Dc are meeting face-to-face in hospital Hsp.

Since the diagnosis-related information about the neuropsychiatric disorder of test subject Sb1 is inferred from the plurality of perspectives, multifaceted analysis of the neuropsychiatric disorder of test subject Sb1 becomes possible. As a result, measures required to improve the neuropsychiatric disorder of test subject Sb1 can be derived appropriately.

Each of schizophrenia inference engine 171, depression inference engine 172 and dementia inference engine 173 may be implemented by a method using an inference model learned with artificial intelligence, machine learning and the like, or may be implemented by a method using a rule-based inference algorithm. In addition, the number of neuropsychiatric disorders inferred by state estimation unit 170 may be one, or may be four or more.

First information output unit 191 outputs the diagnosis-related information from diagnosis unit 130, the intervention information from state acquisition unit 161, the intervention history information from intervention history acquisition unit 162, and the information about the state of the related person of test subject Sb1 from related person information acquisition unit 163 to terminal device 1000 of doctor Dc. By referring to the diagnosis-related information from information processing device 100, doctor Dc can determine whether the word-and-action information of test subject Sb1 in front of doctor Dc is objective. As a result, doctor Dc can perform objective diagnosis of the neuropsychiatric disorder of test subject Sb1.

Second information output unit 192 adds at least one of information about medication guidance to test subject Sb1 (e.g., information about recommendation for a one-time dose medication or information about attention calling for urging test subject Sb1 to take a daily medication as prescribed), information about recommendation for test subject Sb1 to receive a medical examination, and information about recommendation for test subject Sb1 to communicate with the related person to the diagnosis-related information from diagnosis unit 130, and outputs the diagnosis-related information to terminal device 200 of test subject Sb1, terminal device 300 of spouse Rt11 and terminal device 400 of visiting nurse Rt12. Based on the diagnosis-related information from diagnosis unit 130, second information output unit 192 may output warning information to terminal device 200 of test subject Sb1, or may output warning information or information for urging intervention in test subject Sb1 to the terminal device of the related person of test subject Sb1. Second information output unit 192 may have the function of navigation to a medical examination appointment page, the function of emergency notification to doctor Dc or the like, or the function of recommended intervention content notification. When the state of the neuropsychiatric disorder of test subject Sb1 is relatively bad, second information output unit 192 may output a message that actively recommends asking others for assistance (e.g., “may ask others for help”) to terminal device 200 of test subject Sb1.

Output unit 190 may output the diagnosis-related information from diagnosis unit 130 and the recommendation information based on the diagnosis-related information to a not-shown display device such as a display directly connected to information processing device 100.

By referring to the diagnosis-related information from second information output unit 192, test subject Sb1 or the necessary related person (such as, for example, spouse Rt11 or visiting nurse Rt12) can know the measures required to improve the neuropsychiatric disorder of test subject Sb1, based on the presence or absence and the objective state of the neuropsychiatric disorder.

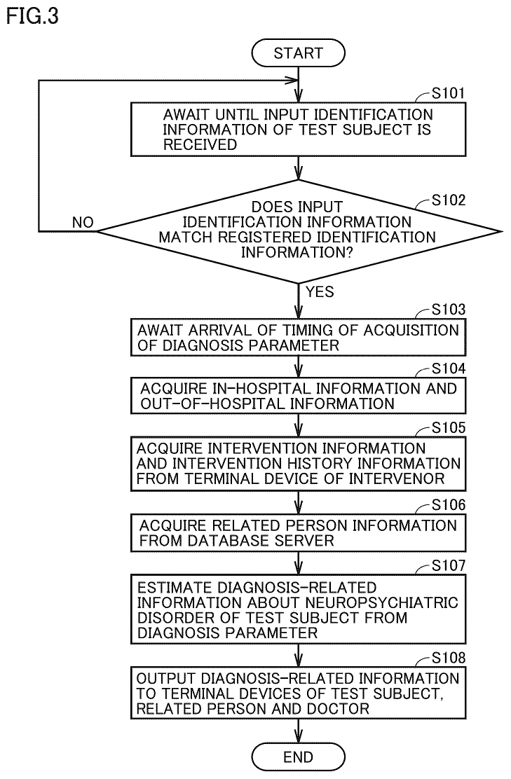

FIG. 3 is a flowchart showing an example of a flow of a process performed by information processing device 100 in FIG. 2. Hereinafter, the step will be simply denoted as “S”.

As shown in FIG. 3, in S101, authentication unit 110 awaits until authentication unit 110 receives the input identification information from the terminal device of the test subject. In response to the reception of the input identification information, authentication unit 110 determines in S102 whether the input identification information matches the registered identification information. When the input identification information does not match the registered identification information (NO in S102), authentication unit 110 returns the process to S101. When the input identification information matches the registered identification information (YES in S102), authentication unit 110 outputs a result of matching between the input identification information and the registered identification information to diagnosis unit 130.

In response to the reception of the result of matching from authentication unit 110, diagnosis unit 130 awaits arrival of the timing of acquisition of the diagnosis parameter in S103. In response to the arrival of the timing of acquisition of the data, diagnosis unit 130 acquires the out-of-hospital information from the terminal device of the test subject authenticated by authentication unit 110 and acquires the in-hospital information from the devices (terminal device 1000 of doctor Dc, camera Cm1 and microphone Mc1) in the hospital which the test subject visits in S104, and causes the process to proceed to S105. In S105, diagnosis unit 130 acquires the intervention information and the intervention history information from the terminal device of the intervenor, and causes the process to proceed to S106. In S106, diagnosis unit 130 acquires the related person information from database server DBS, and causes the process to proceed to S107. In S107, diagnosis unit 130 estimates the diagnosis-related information about the neuropsychiatric disorder of the test subject from the diagnosis parameter, and outputs the diagnosis-related information to output unit 190.

In response to the reception of the diagnosis-related information from diagnosis unit 130, output unit 190 outputs the diagnosis-related information to the terminal devices of the test subject, the related person and the doctor in S108, and ends the process.

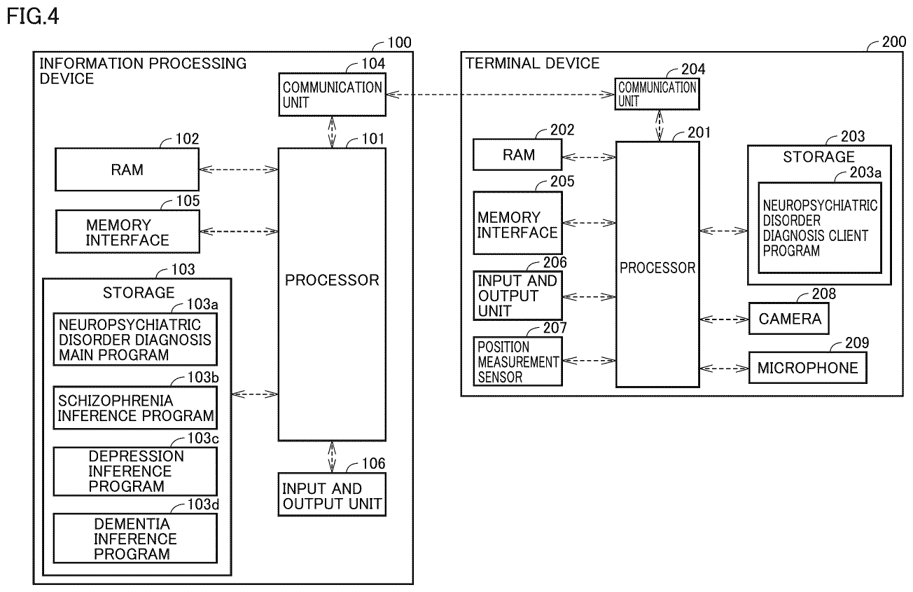

FIG. 4 is a diagram showing an example of a hardware configuration of each of information processing device 100 and terminal device 200 in FIG. 2. As shown in FIG. 4, information processing device 100 includes a processor 101, a random access memory (RAM) 102, a storage 103, a communication unit 104, a memory interface 105, and an input and output unit 106.

Processor 101 is a processing circuit for executing various functions of controlling the operation of information processing device 100. Processor 101 includes, for example, a central processing unit (CPU) or a graphics processing unit (GPU) that causes an information device such as a computer to operate.

RAM 102 includes a volatile memory. RAM 102 is used as a work area when processor 101 operates, as a temporary data storage area, or the like.

Storage 103 includes, for example, a nonvolatile memory such as a flash read only memory (ROM) or a solid state drive (SSD). Computer programs and data to which the computer programs refer at the time of execution of the computer programs are stored in storage 103. For example, an operating system (OS) program (not shown), a neuropsychiatric disorder diagnosis main program 103a, a schizophrenia inference program 103b, a depression inference program 103c, and a dementia inference program 103d are stored in storage 103 as the computer programs. Schizophrenia inference program 103b, depression inference program 103c and dementia inference program 103d are called from neuropsychiatric disorder diagnosis main program 103a. Neuropsychiatric disorder diagnosis main program 103a, schizophrenia inference program 103b, depression inference program 103c, and dementia inference program 103d may be implemented as one program.

The functions of information processing device 100 shown in FIG. 3 are implemented by processor 101 executing neuropsychiatric disorder diagnosis main program 103a. Processor 101 executing schizophrenia inference program 103b corresponds to schizophrenia inference engine 171 in FIG. 2. Processor 101 executing depression inference program 103c corresponds to depression inference engine 172 in FIG. 2. Processor 101 executing dementia inference program 103d corresponds to dementia inference engine 173 in FIG. 2.

Communication unit 104 communicates with terminal device 200 through network NW. Communication unit 104 and network NW may be connected by wired communication (e.g., Ethernet (registered trademark)), or may be connected by wireless communication (e.g., Wi-Fi (registered trademark)).

Memory interface 105 enables access to a storage medium external to information processing device 100. Memory interface 105 corresponds to a secure digital (SD) card and a universal serial bus (USB).

Input and output unit 106 receives an input from a user and displays a result of processing by processor 101 to the user. Input and output unit 106 includes, for example, a display, a touch panel, a keyboard, a speaker, a lamp, and a mouse.

Terminal device 200 includes a processor 201, a RAM 202, a storage 203, a communication unit 204, a memory interface 205, an input and output unit 206, a position measurement sensor 207, a camera 208, and a microphone 209. Since processor 201, RAM 202, communication unit 204, memory interface 205, and input and output unit 206 have substantially the same functions as those of processor 101, RAM 102, communication unit 104, memory interface 105, and input and output unit 106 of information processing device 100, respectively, description about the configurations of these components of terminal device 200 will not be repeated. Each of terminal devices 300 and 400 of related persons Rt11 and Rt12 also has the same hardware configuration as that of terminal device 200.

Camera 208 acquires image data in the surroundings of terminal device 200 and stores the image data in storage 203. Microphone 209 acquires speech data in the surroundings of terminal device 200 and stores the speech data in storage 203. Position measurement sensor 207 acquires position data of terminal device 200 using a global navigation satellite system (GNSS) such as a global positioning system (GPS), and stores the position data in storage 203. The image data, the speech data and the position data stored in storage 203 are transmitted to information processing device 100 by communication unit 204.

An OS program (not shown) and a neuropsychiatric disorder diagnosis client program 203a are stored in storage 203. Processor 201 executing neuropsychiatric disorder diagnosis client program 203a controls terminal device 200 to cooperate with the information processing device executing neuropsychiatric disorder diagnosis main program 103a.

As described above, according to the information processing device, the system, the program, and the method of the first embodiment, objective diagnosis and state evaluation relating to the neuropsychiatric disorder of the subject can be assisted.

### Second Embodiment

In the first embodiment, the configuration in which the diagnosis-related information about the neuropsychiatric disorder of the test subject is estimated using the out-of-hospital information and the in-hospital information of the test subject has been described. In a second embodiment, a configuration in which the diagnosis-related information about the neuropsychiatric disorder of the test subject is estimated using word-and-action information of the test subject in a virtual space (virtual space information) and word-and-action information of the test subject in a real space (real space information) will be described.

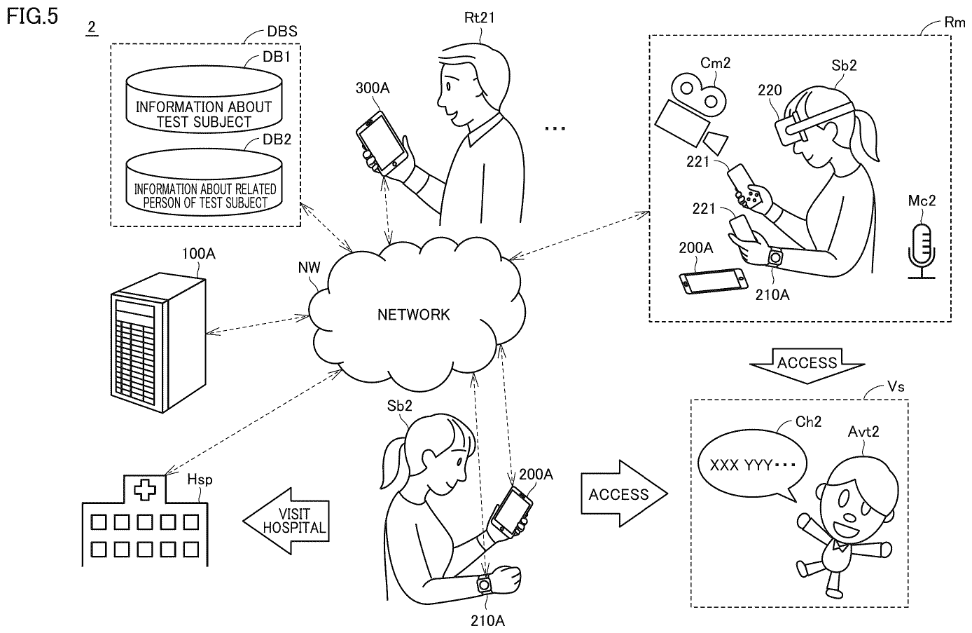

FIG. 5 is a block diagram showing a configuration of a neuropsychiatric disorder diagnosis system 2 according to the second embodiment. In FIG. 5, a test subject of neuropsychiatric disorder diagnosis system 2 is denoted as Sb2, and a related person of test subject Sb2 is denoted as Rt21. In addition, information processing device 100 and terminal devices 200, 210 and 300 in FIG. 1 are replaced with an information processing device 100A and terminal devices 200A, 210A and 300A, respectively. In addition, since hospital Hsp, database server DBS and network NW in FIG. 5 are the same as hospital Hsp, database server DBS and network NW in FIG. 1, respectively, description thereof will not be repeated.

As shown in FIG. 5, terminal device 200A of test subject Sb2 acquires word-and-action information of test subject Sb2, and transmits the word-and-action information to information processing device 100A together with time information. Terminal device 200A includes, for example, a smartphone, a tablet and a personal computer. Information about a behavior history acquired by terminal device 200A includes, for example, an operation time of terminal device 200A per day, a purchase history (e.g., the number of purchases and a total purchase amount) in Internet shopping per week, a movement range of terminal device 200A, or a text input to a search engine, a social networking service (SNS) or an electronic mail. Speech information acquired by terminal device 200A includes, for example, text information of speech conversation of test subject Sb2. A cycle of sampling and a timing of acquisition of the word-and-action information using terminal device 200A of test subject Sb2 may be arbitrarily set.

Terminal device 210A of test subject Sb2 includes a biological information sensor (e.g., a pulse sensor). Terminal device 210A acquires biological information of test subject Sb2 through the biological information sensor, and transmits the biological information to information processing device 100A together with time information. Terminal device 210A includes, for example, a wearable smart watch. A cycle of sampling and a timing of acquisition of the biological information using terminal device 210A of test subject Sb2 may be arbitrarily set.

Terminal device 200A of test subject Sb2 has an application (e.g., a game) for accessing a virtual space Vs installed thereon. Virtual space Vs may be a space of the game formed in terminal device 200A, or may be a virtual space (e.g., a metaverse) formed on a cloud. Through terminal device 200A, test subject Sb2 operates an avatar Avt2 that can do activities in virtual space Vs. Test subject Sb2 can input a chat Ch2 of avatar Avt2 through terminal device 200A and interact with another player. Test subject Sb2 may access virtual space Vs formed as virtual reality. For example, test subject Sb2 wears a head mounted display 220 in a room Rm at home to immerse himself/herself in virtual space Vs and operate avatar Avt2 through a controller 221.

Since the personal identification information such as name and face can be kept secret in virtual space Vs, virtual space Vs is higher in anonymity than a real space. Therefore, an objective state of test subject Sb2 is more likely to be expressed unconsciously in the word-and-action information of test subject Sb2 in virtual space Vs than that in the real space. Thus, neuropsychiatric disorder diagnosis system 2 estimates the diagnosis-related information about the neuropsychiatric disorder using the real space information and the virtual space information of test subject Sb2. According to neuropsychiatric disorder diagnosis system 2, objective diagnosis and state evaluation relating to the neuropsychiatric disorder of test subject Sb2 can be assisted.

Terminal device 200A transmits word-and-action information of test subject Sb2 when test subject Sb2 is accessing virtual space Vs to information processing device 100A together with time information. Information about a behavior history when test subject Sb2 is accessing virtual space Vs includes a login time period for which test subject Sb2 is accessing virtual space Vs, a behavior range in virtual space Vs, and a content of an activity in virtual space Vs. Speech information when test subject Sb2 is accessing virtual space Vs includes a content of a chat in virtual space Vs. A camera Cm2 and a microphone Mc2 placed in room Rm acquire image data and speech data of test subject Sb2 when test subject Sb2 is accessing virtual space Vs, respectively, and transmit the image data and the speech data to information processing device 100A together with time information.

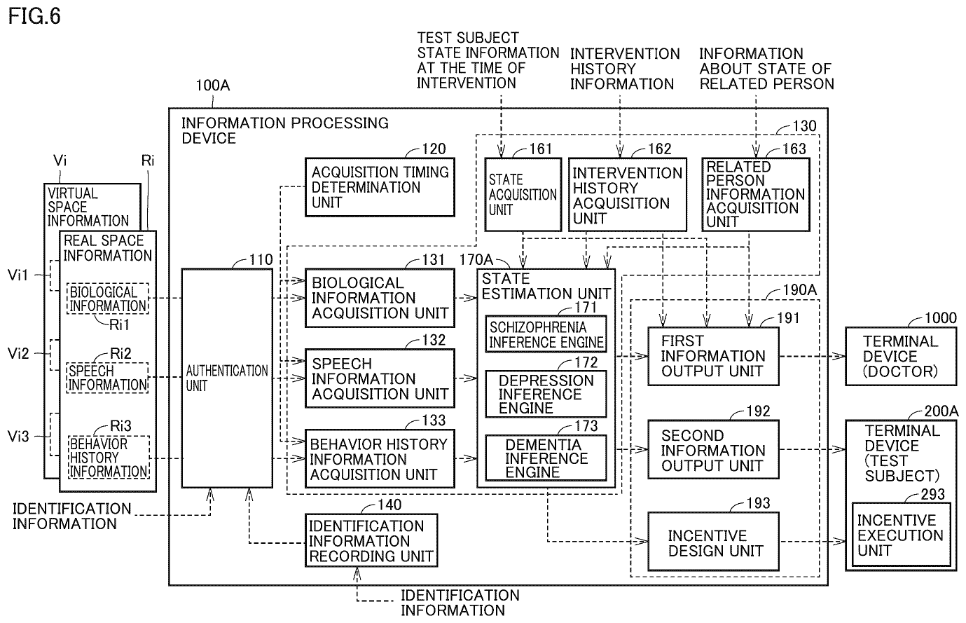

FIG. 6 is a block diagram showing a functional configuration of information processing device 100A in FIG. 5. In the configuration of information processing device 100A, state estimation unit 170 and output unit 190 in FIG. 2 are replaced with a state estimation unit 170A and an output unit 190A, respectively. Output unit 190A is configured by adding an incentive design unit 193 to output unit 190 in FIG. 2. Since the remaining configuration of information processing device 100A is the same as the configuration of information processing device 100, description about the same configuration will not be repeated.

As shown in FIG. 6, information processing device 100A receives real space information Ri and virtual space information Vi. Virtual space information Vi includes biological information Vi1, speech information Vi2 and behavior history information Vi3 of test subject Sb2. Real space information Ri includes biological information Ri1, speech information Ri2 and behavior history information Ri3 of test subject Sb2. Real space information Ri may include at least one of the out-of-hospital information and the in-hospital information.

State estimation unit 170A determines the state of test subject Sb2 and captures a change on the space in which test subject Sb2 is interested, by analyzing a difference and a degree of deviation between the word-and-action information of test subject Sb2 in the real space and the word-and-action information of test subject Sb2 in the virtual space. The word-and-action information of test subject Sb2 in the real space may include word-and-action information of test subject Sb2 when test subject Sb2 and a doctor are meeting face-to-face in hospital Hsp.

In accordance with the diagnosis-related information from diagnosis unit 130, incentive design unit 193 designs incentive information that motivates treatment of the neuropsychiatric disorder of test subject Sb2, and outputs the incentive information to terminal device 200A. The incentive may be designed as, for example, a game element in virtual space Vs, such as growing avatar Avt2 to change an appearance thereof, in order to gamify continuation of treatment of the neuropsychiatric disorder (gamification).

Terminal device 200A includes an incentive execution unit 293. Incentive execution unit 293 reflects the incentive information from information processing device 100A in the activity of test subject Sb2 (e.g., the appearance of avatar Avt2) in virtual space Vs.

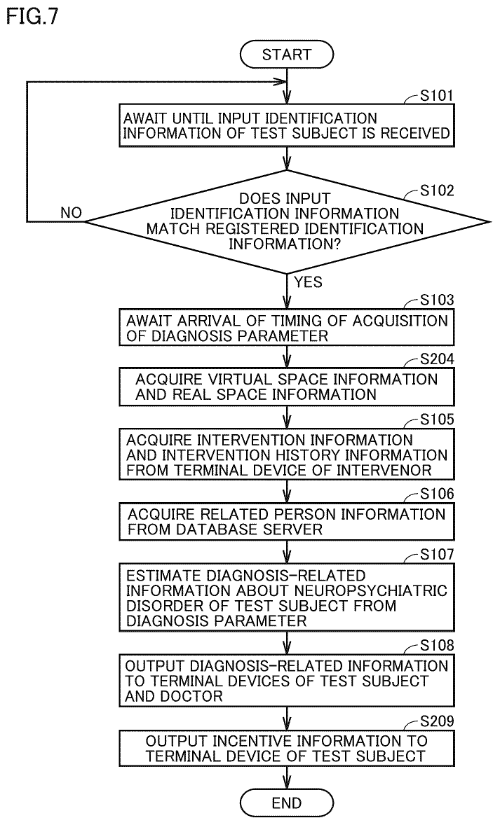

FIG. 7 is a flowchart showing an example of a flow of a process performed by information processing device 100A in FIG. 6. In the flowchart shown in FIG. 7, S104 in FIG. 3 is replaced with S204 and S209 is added. Description about the same processing as that in FIG. 3 will not be repeated.

As shown in FIG. 7, after S101 to S103 are performed similarly to the first embodiment, diagnosis unit 130 acquires the virtual space information and the real space information from the terminal device of the test subject authenticated by authentication unit 110 in S204 in response to the arrival of the timing of acquisition of the data, and causes the process to proceed to S105. Diagnosis unit 130 may acquire the image data and the speech data as the virtual space information from the camera and the microphone placed in the room where the test subject accesses the virtual space. Diagnosis unit 130 may acquire the in-hospital information as the real space information from the devices in the hospital which the test subject visits. After S105 to S108 are performed similarly to the first embodiment, output unit 190A outputs the incentive information to the terminal device of the test subject in S209, and ends the process.

As described above, according to the information processing device, the system, the program, and the method of the second embodiment, objective diagnosis and state evaluation relating to the neuropsychiatric disorder of the subject can be assisted.

### Additional Notes

The embodiments according to the present invention described above include the following configurations.

### Configuration 1

An information processing device that estimates diagnosis-related information about a neuropsychiatric disorder of a subject, the information processing device comprising:
a diagnosis unit; and
an output unit, wherein
the diagnosis unit estimates the diagnosis-related information from a diagnosis parameter in at least two of a first case, a second case and a third case,
the output unit outputs the diagnosis-related information estimated by the diagnosis unit to the outside of the information processing device,
the diagnosis parameter includes word-and-action information of the subject,
the word-and-action information of the subject includes at least one of speech information and a behavior history of the subject,
the first case is a case in which the subject is outside a specific facility where the subject and a doctor meet face-to-face for a medical examination of the neuropsychiatric disorder by the doctor, and in which the subject is not accessing a virtual space,
the second case is a case in which the subject and the doctor are meeting face-to-face in the specific facility, and
the third case is a case in which the subject is accessing the virtual space.

### Configuration 2

The information processing device according to Configuration 1, wherein
the diagnosis parameter uses the first case and the second case.

### Configuration 3

The information processing device according to Configuration 1 or 2, wherein
the word-and-action information of the subject is the speech information and the behavior history.

### Configuration 4

The information processing device according to any one of Configurations 1 to 3, wherein
the diagnosis parameter further includes biological information of the subject.

### Configuration 5

The information processing device according to any one of Configurations 1 to 4, wherein
the diagnosis parameter further includes a degree of deviation between word-and-action information of the subject in the first case and word-and-action information of the subject in the second case.

### Configuration 6

The information processing device according to Configuration 1, wherein
the diagnosis parameter uses at least one of the first case and the second case, and the third case.

### Configuration 7

The information processing device according to Configuration 6, wherein
the word-and-action information of the subject is the speech information and the behavior history.

### Configuration 8

The information processing device according to Configuration 6 or 7, wherein
the diagnosis parameter further includes biological information of the subject.

### Configuration 9

The information processing device according to any one of Configurations 6 to 8, wherein
the diagnosis parameter further includes login time period information for which the subject is accessing the virtual space.

### Configuration 10

The information processing device according to any one of Configurations 6 to 9, wherein
the diagnosis parameter further includes at least one of a behavior history of a related person of the subject and biological information of the related person when the related person is accessing the virtual space.

### Configuration 11

The information processing device according to any one of Configurations 6 to 10, wherein
the diagnosis parameter further includes at least any one of a degree of deviation between word-and-action information of the subject in the first case and word-and-action information of the subject in the second case, a degree of deviation between the word-and-action information of the subject in the second case and word-and-action information of the subject in the third case, and a degree of deviation between the word-and-action information of the subject in the first case and the word-and-action information of the subject in the third case.

### Configuration 12

The information processing device according to any one of Configurations 6 to 11, wherein
the output unit outputs incentive information to a terminal device in accordance with the diagnosis-related information, the incentive information being information for changing an appearance of an avatar operated in the virtual space by the subject through the terminal device.

### Configuration 13

The information processing device according to any one of Configurations 1 to 12, wherein
the diagnosis-related information includes information about a plurality of neuropsychiatric disorders different from each other.

### Configuration 14

The information processing device according to any one of Configurations 1 to 13, wherein
the diagnosis parameter further includes at least one of information about a state of the subject and information about a related person of the subject when the related person intervenes in the subject.

### Configuration 15

The information processing device according to Configuration 14, wherein
the output unit further outputs the information about the state of the subject when the related person of the subject intervenes in the subject to the outside of the information processing device.

### Configuration 16

The information processing device according to Configuration 14 or 15, wherein
the diagnosis parameter further includes at least one of a history of intervention in the subject by the related person, the state of the subject when the related person intervenes in the subject, information about a medicine-taking situation of the subject, information about whether a home-visit care service has been provided to the subject, information about a neuropsychiatric state of the subject when the home-visit care service has been provided, information about a trigger event when the neuropsychiatric state of the subject improves or worsens, and information about a neuropsychiatric disorder of the related person of the subject.

### Configuration 17

The information processing device according to any one of Configurations 1 to 16, wherein
the diagnosis-related information includes at least one of warning information to the subject, warning information to a related person of the subject, information about medication guidance to the subject, information about recommendation for the subject to receive a medical examination, and information about recommendation for the subject to communicate with the related person.

### Configuration 18

The information processing device according to any one of Configurations 1 to 17, further comprising an authentication unit, wherein
the authentication unit determines whether input identification information input to identify the subject matches registered identification information of the subject registered in advance, and
the diagnosis unit estimates the diagnosis-related information from the diagnosis parameter when the authentication unit determines that the input identification information matches the registered identification information.

### Configuration 19

The information processing device according to any one of Configurations 1 to 18, further comprising an acquisition timing determination unit, wherein
the acquisition timing determination unit determines a timing of acquisition of the diagnosis parameter, and
the diagnosis unit estimates the diagnosis-related information using the diagnosis parameter acquired at the timing of acquisition.

### Configuration 20

A system that estimates diagnosis-related information about a neuropsychiatric disorder of a subject, the system comprising:
at least one word-and-action monitoring device that acquires word-and-action information of the subject;
a terminal device; and
an information processing device that estimates the diagnosis-related information from a diagnosis parameter and transmits information about the diagnosis-related information to the terminal device, wherein
the diagnosis parameter includes the word-and-action information of the subject acquired by the at least one word-and-action monitoring device,
the word-and-action information of the subject includes at least one of speech information and a behavior history of the subject,
the diagnosis parameter includes word-and-action information of the subject in at least two of a first case, a second case and a third case,
the first case is a case in which the subject is outside a specific facility where the subject and a doctor meet face-to-face for a medical examination of the neuropsychiatric disorder by the doctor, and in which the subject is not accessing a virtual space,
the second case is a case in which the subject and the doctor are meeting face-to-face in the specific facility, and
the third case is a case in which the subject is accessing the virtual space.

### Configuration 21

A program for estimating diagnosis-related information about a neuropsychiatric disorder of a subject, wherein
by being executed by a processor, the program estimates the diagnosis-related information from a diagnosis parameter in at least two of a first case, a second case and a third case, and transmits information about the diagnosis-related information to a terminal device,
the diagnosis parameter includes word-and-action information of the subject,
the word-and-action information of the subject includes at least one of speech information and a behavior history of the subject,
the first case is a case in which the subject is outside a specific facility where the subject and a doctor meet face-to-face for a medical examination of the neuropsychiatric disorder by the doctor, and in which the subject is not accessing a virtual space,
the second case is a case in which the subject and the doctor are meeting face-to-face in the specific facility, and
the third case is a case in which the subject is accessing the virtual space.

### Configuration 22

A method for estimating diagnosis-related information about a neuropsychiatric disorder of a subject, the method comprising:
estimating the diagnosis-related information from a diagnosis parameter in at least two of a first case, a second case and a third case; and
outputting information about the diagnosis-related information to a terminal device, wherein
the diagnosis parameter includes word-and-action information of the subject,
the word-and-action information of the subject includes at least one of speech information and a behavior history of the subject,
the first case is a case in which the subject is outside a specific facility where the subject and a doctor meet face-to-face for a medical examination of the neuropsychiatric disorder by the doctor, and in which the subject is not accessing a virtual space,
the second case is a case in which the subject and the doctor are meeting face-to-face in the specific facility, and
the third case is a case in which the subject is accessing the virtual space.

The embodiments disclosed herein are also intended to be carried out as being combined as appropriate within the consistent scope. It should be understood that the embodiments disclosed herein are illustrative and non-restrictive in every respect. The scope of the present invention is defined by the terms of the claims, rather than the description above, and is intended to include any modifications within the scope and meaning equivalent to the terms of the claims.

### REFERENCE SIGNS LIST

1, 2 neuropsychiatric disorder diagnosis system; 100, 100A information processing device; 101, 201 processor; 102, 202 RAM; 103, 203 storage, 103a neuropsychiatric disorder diagnosis main program; 103b schizophrenia inference program; 103c depression inference program; 103d dementia inference program; 104, 204 communication unit; 105, 205 memory interface; 106, 206 input and output unit; 110 authentication unit; 120 acquisition timing determination unit; 130 diagnosis unit; 131 biological information acquisition unit; 132 speech information acquisition unit; 133 behavior history information acquisition unit; 140 identification information recording unit; 150 behavior history acquisition unit; 161 state acquisition unit; 162 intervention history acquisition unit; 163 related person information acquisition unit; 170, 170A state estimation unit; 171 schizophrenia inference engine, 172 depression inference engine; 173 dementia inference engine; 190, 190A output unit; 191 first information output unit; 192 second information output unit; 193 incentive design unit; 200, 200A, 210, 210A, 300, 300A, 400, 1000 terminal device; 203a neuropsychiatric disorder diagnosis client program; 207 position measurement sensor; 208, Cm1, Cm2 camera; 209, Mc1, Mc2 microphone; 220 head mounted display; 221 controller; 293 incentive execution unit; Avt2 avatar; Ch2 chat; DB1, DB2 database; DBS database server; Dc doctor; Hi1, Ho1, Ri1, Vi1 biological information; Hi2, Ho2, Ri2, Vi2 speech information; Hi3, Ho3, Ri3, Vi3 behavior history information; Hin in-hospital information; Hout out-of-hospital information; Hsp hospital; NW network; Ri real space information; Rm room; Rt11, Rt12, Rt21 related person; Sb1, Sb2 test subject; Vi virtual space information; Vs virtual space.

1. An information processing device that estimates diagnosis-related information about a neuropsychiatric disorder of a subject, the information processing device comprising:
a diagnosis unit; and
an output unit, wherein
the diagnosis unit estimates the diagnosis-related information from a diagnosis parameter in at least two of a first case, a second case and a third case,
the output unit outputs the diagnosis-related information estimated by the diagnosis unit to the outside of the information processing device,
the diagnosis parameter includes word-and-action information of the subject,
the word-and-action information of the subject includes at least one of speech information and a behavior history of the subject,
the first case is a case in which the subject is outside a specific facility where the subject and a doctor meet face-to-face for a medical examination of the neuropsychiatric disorder by the doctor, and in which the subject is not accessing a virtual space,
the second case is a case in which the subject and the doctor are meeting face-to-face in the specific facility, and
the third case is a case in which the subject is accessing the virtual space.

2. The information processing device according to claim 1, wherein the diagnosis parameter uses the first case and the second case.

3. The information processing device according to claim 1, wherein the word-and-action information of the subject is the speech information and the behavior history.

4. The information processing device according to claim 1, wherein the diagnosis parameter further includes biological information of the subject.

5. The information processing device according to claim 1, wherein the diagnosis parameter further includes a degree of deviation between word-and-action information of the subject in the first case and word-and-action information of the subject in the second case.

6. The information processing device according to claim 1, wherein the diagnosis parameter uses at least one of the first case and the second case, and the third case.

7. The information processing device according to claim 6, wherein the word-and-action information of the subject is the speech information and the behavior history.

8. The information processing device according to claim 6, wherein the diagnosis parameter further includes biological information of the subject.

9. The information processing device according to claim 6, wherein the diagnosis parameter further includes login time period information for which the subject is accessing the virtual space.

10. The information processing device according to claim 6, wherein the diagnosis parameter further includes at least one of a behavior history of a related person of the subject and biological information of the related person when the related person is accessing the virtual space.

11. The information processing device according to claim 6, wherein the diagnosis parameter further includes at least any one of a degree of deviation between word-and-action information of the subject in the first case and word-and-action information of the subject in the second case, a degree of deviation between the word-and-action information of the subject in the second case and word-and-action information of the subject in the third case, and a degree of deviation between the word-and-action information of the subject in the first case and the word-and-action information of the subject in the third case.

12. The information processing device according to claim 6, wherein the output unit outputs incentive information to a terminal device in accordance with the diagnosis-related information, the incentive information being information for changing an appearance of an avatar operated in the virtual space by the subject through the terminal device.

13. The information processing device according to claim 1, wherein the diagnosis-related information includes information about a plurality of neuropsychiatric disorders different from each other.

14. The information processing device according to claim 1, wherein the diagnosis parameter further includes at least one of information about a state of the subject and information about a related person of the subject when the related person intervenes in the subject.

15. The information processing device according to claim 14, wherein the output unit further outputs the information about the state of the subject when the related person of the subject intervenes in the subject to the outside of the information processing device.

16. The information processing device according to claim 14, wherein the diagnosis parameter further includes at least one of a history of intervention in the subject by the related person, the state of the subject when the related person intervenes in the subject, information about a medicine-taking situation of the subject, information about whether a home-visit care service has been provided to the subject, information about a neuropsychiatric state of the subject when the home-visit care service has been provided, information about a trigger event when the neuropsychiatric state of the subject improves or worsens, and information about a neuropsychiatric disorder of the related person of the subject.

17. The information processing device according to claim 1, wherein the diagnosis-related information includes at least one of warning information to the subject, warning information to a related person of the subject, information about medication guidance to the subject, information about recommendation for the subject to receive a medical examination, and information about recommendation for the subject to communicate with the related person.

18. The information processing device according to claim 1, further comprising an authentication unit, wherein:
the authentication unit determines whether input identification information input to identify the subject matches registered identification information of the subject registered in advance, and
the diagnosis unit estimates the diagnosis-related information from the diagnosis parameter when the authentication unit determines that the input identification information matches the registered identification information.

19. The information processing device according to claim 1, further comprising an acquisition timing determination unit, wherein:
the acquisition timing determination unit determines a timing of acquisition of the diagnosis parameter, and
the diagnosis unit estimates the diagnosis-related information using the diagnosis parameter acquired at the timing of acquisition.

20. A system that estimates diagnosis-related information about a neuropsychiatric disorder of a subject, the system comprising:
at least one word-and-action monitoring device that acquires word-and-action information of the subject;
a terminal device; and
an information processing device that estimates the diagnosis-related information from a diagnosis parameter and transmits information about the diagnosis-related information to the terminal device, wherein
the diagnosis parameter includes the word-and-action information of the subject acquired by the at least one word-and-action monitoring device,
the word-and-action information of the subject includes at least one of speech information and a behavior history of the subject,
the diagnosis parameter includes word-and-action information of the subject in at least two of a first case, a second case and a third case,
the first case is a case in which the subject is outside a specific facility where the subject and a doctor meet face-to-face for a medical examination of the neuropsychiatric disorder by the doctor, and in which the subject is not accessing a virtual space,
the second case is a case in which the subject and the doctor are meeting face-to-face in the specific facility, and
the third case is a case in which the subject is accessing the virtual space.

21. A program for estimating diagnosis-related information about a neuropsychiatric disorder of a subject, wherein:
by being executed by a processor, the program estimates the diagnosis-related information from a diagnosis parameter in at least two of a first case, a second case and a third case, and transmits information about the diagnosis-related information to a terminal device,
the diagnosis parameter includes word-and-action information of the subject,
the word-and-action information of the subject includes at least one of speech information and a behavior history of the subject,
the first case is a case in which the subject is outside a specific facility where the subject and a doctor meet face-to-face for a medical examination of the neuropsychiatric disorder by the doctor, and in which the subject is not accessing a virtual space,
the second case is a case in which the subject and the doctor are meeting face-to-face in the specific facility, and
the third case is a case in which the subject is accessing the virtual space.

22. A method for estimating diagnosis-related information about a neuropsychiatric disorder of a subject, the method comprising:
estimating the diagnosis-related information from a diagnosis parameter in at least two of a first case, a second case and a third case; and
outputting information about the diagnosis-related information to a terminal device, wherein
the diagnosis parameter includes word-and-action information of the subject,
the word-and-action information of the subject includes at least one of speech information and a behavior history of the subject,
the first case is a case in which the subject is outside a specific facility where the subject and a doctor meet face-to-face for a medical examination of the neuropsychiatric disorder by the doctor, and in which the subject is not accessing a virtual space,
the second case is a case in which the subject and the doctor are meeting face-to-face in the specific facility, and
the third case is a case in which the subject is accessing the virtual space.
